(19) United States
(12) Patent Application Publication
(10) Pub. No.: US 2023/0414111 A1
(43) Pub. Date: Dec. 28, 2023

(54) METHODS AND SYSTEMS FOR MEDICAL IMAGE PROCESSING

(71) Applicant: WUHAN UNITED IMAGING HEALTHCARE CO., LTD., Wuhan (CN)

(72) Inventor: Jian SUN, Wuhan (CN)

(73) Assignee: WUHAN UNITED IMAGING HEALTHCARE CO., LTD., Wuhan (CN)

(21) Appl. No.: 18/349,899

(22) Filed: Jul. 10, 2023

Related U.S. Application Data
(63) Continuation of application No. PCT/CN2023/076362, filed on Feb. 16, 2023.

(30) Foreign Application Priority Data
Jun. 27, 2022 (CN) 202210745505.5

(51) Int. Cl. A61B 5/01; G06T 7/00; G06V 10/56

(52) U.S. Cl. CPC A61B 5/015; G06T 7/0012; G06V 10/56; G06T 2207/10048; G06T 2207/10081; G06T 2207/10088; G06T 2207/10132

(57) ABSTRACT

The embodiments of the present disclosure provide methods and systems for medical image processing. The method may include obtaining a first image of a target object; obtaining a target mapping relationship; obtaining a target temperature parameter by performing a first processing on the first image based on the target mapping relationship; and determining a target image based on the target temperature parameter and the first image.

### CROSS-REFERENCE TO RELATED APPLICATIONS

This application is a Continuation of International Application No. PCT/CN2023/076362, filed on Feb. 16, 2023, which claims priority to Chinese Application No. 202210745505.5 filed on Jun. 27, 2022, the entire contents of each of which are incorporated herein by reference.

### TECHNICAL FIELD

The present application relates to the field of medical imaging technology, and in particular, to methods and systems for medical image processing.

### BACKGROUND

Medical images are images that reflect the internal structure of the human body and are one of the main bases of modern medical diagnosis. The manners of medical image detection include ultrasound detection, magnetic resonance (MR) detection, computed tomography (CT) detection, etc. If a single image detection manner is used for medical diagnosis, due to objects to be detected being different, detection images may be not clear enough and a lesion is not accurately located enough to meet the diagnosis needs. Taking ultrasound detection as an example, for a single piece of evidence for a diagnosis of a specific disease, especially when the lesion is too small or the acoustic impedance is too large to cause an emission, the effect of ultrasound detection would be affected.

Therefore, it is desirable to provide methods and systems for medical images processing, which gives the play of advantages of medical detection and also makes full use of a reference role of infrared thermometry for lesion determination, thereby effectively improving the accuracy of medical diagnosis.

### SUMMARY

According to one of the embodiments of the present disclosure, a method for medical image processing is provided. The method may include obtaining a first image of a target object; obtaining a target mapping relationship; obtaining a target temperature parameter by performing a first processing on the first image based on the target mapping relationship; and determining a target image based on the target temperature parameter and the first image.

According to one of the embodiments of the present disclosure, a method for ultrasound image processing is provided. The method may include obtaining an ultrasound image of an object to be detected; obtaining a temperature parameter corresponding to a second pixel point corresponding to the ultrasound image based on an ultrasound echo model including a temperature feature and a second gray value of the second pixel point; and updating the ultrasound image by assigning a value to the second pixel point based on the temperature parameter.

According to one of the embodiments of the present disclosure, a system for ultrasound image processing is provided. The system may include an obtaining module configured to obtain the first image of the target object, and obtain the target temperature by performing the first processing on the first image based on the target mapping relationship. The system may also include a determination module configured to determine the target image based on the target temperature parameter and the first image.

Beneficial effects of the embodiments of the present disclosure may include at least that: (1) the target image may be obtained by assigning the temperature parameter to a corresponding pixel point in the medical image, which can make the target image compositely display the temperature data of the target object on the basis of a display of the medical detected data. At a same time, an organic combination of an infrared thermal image and a medical image is realized by obtaining a fitting curve of temperature and grayscale, which can make full use of the reference role of an infrared thermometry for a lesion determination while taking advantage of medical detection, and assist in determination by combining relative temperature information on the basis of a traditional determination of lesion or lesion tissues, thereby in turn improving the accuracy of a medical diagnosis, especially providing a supportive analysis evidence for the medical diagnosis of very small or fuzzy lesions and lesion tissues; (2) by respectively displaying the medical image before overlaying the temperature data and the medical image after overlaying the temperature data, a doctor is further be assisted in diagnosis, which can effectively improve a diagnostic efficiency; (3) when a temperature difference is not obvious with the normal tissue, it is difficult for the doctor to identify an abnormal region through the naked eye to further diagnose a lesion condition. However, by a model identification, the region that has an abnormal temperature can be quickly and accurately determined, which is helpful for the doctor to make a further diagnosis and improve diagnosis efficiency effectively; (4) when the lesion is difficult to detect, after the temperature information is introduced on the basis of a traditional ultrasound detection, MR detection information or CT detection information may be further introduced. Then, a fusion image including the temperature data, the ultrasound detection data, and the MR detection data or the CT detection data of the target object can be obtained by organically combining the ultrasound image that overlays the temperature data with an MR image or a CT image, which can further assist the doctor in analyzing and determining the lesion more accurately and effectively for diagnosis. Meanwhile, an ultrasound-MR image fusion technology combines the advantages of ultrasound and MRI, which can display multi-layer MR cross-sectional images associated with the ultrasound image in a clear and real-time manner. In addition, the ultrasound-CT image fusion technology combines the advantages of ultrasound and CT, which can display a CT image associated with the ultrasound image in a clear and real-time manner and provide corresponding technical support for the accuracy of interventional therapy.

### BRIEF DESCRIPTION OF THE DRAWINGS

The present disclosure is further described in terms of exemplary embodiments. The exemplary embodiments are described in detail with reference to the drawings. The embodiments are non-limiting exemplary embodiments, in which like reference numerals represent similar structures throughout the several views of the drawings, and wherein:

FIG. 1 is a schematic diagram illustrating an application scenario of an exemplary system for medical image processing according to some embodiments of the present disclosure;

FIG. 2 is a block diagram illustrating a structure of an exemplary system for medical image processing according to some embodiments of the present description;

FIG. 3 is a flowchart illustrating an exemplary process for medical image processing according to some embodiments of the present disclosure;

FIG. 4 is a flowchart of an exemplary process for obtaining a target mapping relationship according to some embodiments of the present disclosure;

FIG. 5 is a flowchart illustrating an exemplary process for ultrasound image processing according to some embodiments of the present disclosure;

FIG. 6 is a schematic diagram illustrating an application of an exemplary process for ultrasound image processing according to some embodiments of the present disclosure;

FIG. 7 is a flowchart illustrating an exemplary process for obtaining an ultrasound echo model including a temperature feature according to some embodiments of the present disclosure;

FIG. 8 is a schematic diagram illustrating a basic process for measuring an infrared thermometry of an exemplary object to be detected according to some embodiments of the present disclosure;

FIG. 9 is a schematic diagram illustrating a basic process for ultrasound imaging of an exemplary object to be detected according to some embodiments of the present disclosure;

FIG. 10 is a schematic diagram illustrating a process for obtaining an exemplary ultrasound echo model including a temperature feature according to some embodiments of the present disclosure;

FIG. 11 is a schematic diagram illustrating modules of an exemplary obtaining system including an ultrasound echo model including a temperature feature according to some embodiments of the present disclosure;

FIG. 12 is a schematic diagram illustrating an application effect of an exemplary process for ultrasound image processing according to some embodiments of the present disclosure; and

FIG. 13 is a schematic diagram illustrating modules of an exemplary system for ultrasound image processing according to some embodiments of the present disclosure.

### DETAILED DESCRIPTION

To more clearly illustrate the technical solutions of the embodiments of the present disclosure, the accompanying drawings that need to be used in the description of the embodiments would be briefly introduced below. Obviously, the accompanying drawings in the following description are merely some examples or embodiments of the present disclosure, and those skilled in the art can apply the present disclosure to other similar situations according to the drawings without any creative effort. Unless obviously obtained from the context or the context illustrates otherwise, the same numeral in the drawings indicates the same structure or operation.

It will be understood that the terms “system,” “device,” “unit,” and/or “module” used herein are used to distinguish different components, elements, parts, sections, or assemblies of different levels. However, the terms may be displaced by other expressions if they can achieve the same purpose.

As used in the present disclosure and the appended claims, the singular forms “a,” “an,” and “the” are intended to include plural referents, unless the content clearly dictates otherwise. Generally, the terms “comprise” and “include” only imply that the clearly identified steps and elements are included, but these steps and elements may not constitute an exclusive list, and the method or device may further include other steps or elements.

The flowcharts used in the present disclosure illustrate operations that systems implement according to some embodiments of the present disclosure. It is to be expressly understood, the operations of the flowcharts may be implemented not in order. Conversely, the operations may be implemented in an inverted order, or simultaneously. Moreover, one or more other operations may be added to the flowcharts. One or more operations may be removed from the flowcharts.

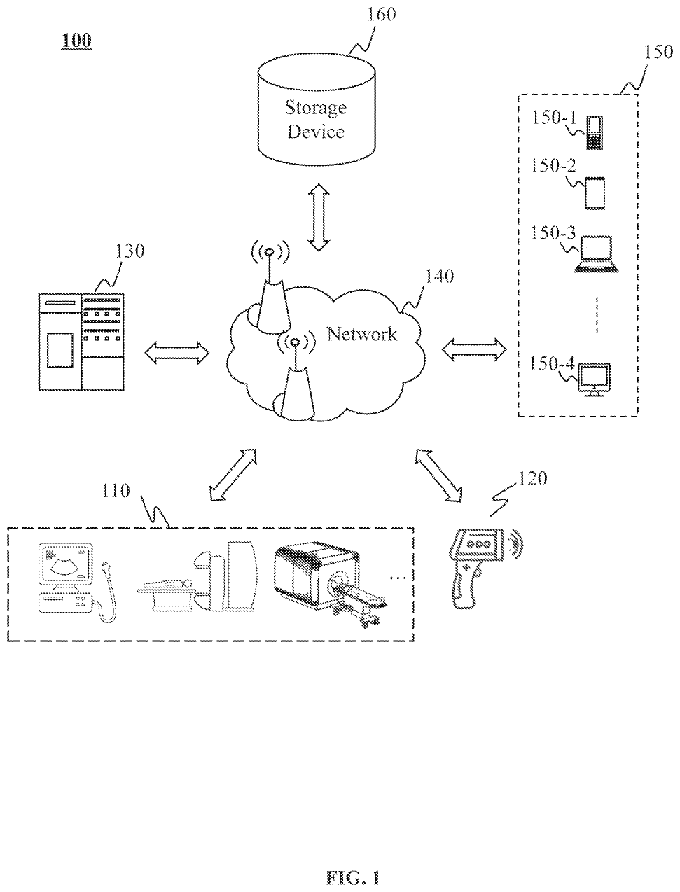

FIG. 1 is a schematic diagram illustrating an application scenario of an exemplary system for medical image processing according to some embodiments of the present disclosure.

In some embodiments, as shown in FIG. 1, a system 100 for medical image processing may include a medical imaging device 110, an infrared thermometric device 120, a processing device 130, a network 140, a terminal device 150, and a storage device 160. In some embodiments, components of the system 100 for medical image processing may be connected to each other through the network 140. In some embodiments, some components in the system 100 for medical image processing may be directly connected to each other.

The medical imaging device 110 may be configured to image an object. In some embodiments, the object may include a living organism, an artificial object, etc. In some embodiments, the object may include a specific part of a body, e.g., the head, the neck, the chest, or the like, or any combination thereof. In some embodiments, the object may include a specific organ, e.g., the liver, the kidney, the pancreas, the bladder, the uterus, or the like, or any combination thereof. In some embodiments, the medical imaging device 110 may include an ultrasound imaging device, an MRI device, a CT device, or the like, or any combination thereof. In some embodiments, the ultrasound imaging device may include an A-type ultrasound diagnostic instrument, an M-type ultrasound diagnostic instrument, a B-type ultrasound diagnostic instrument, a D-type ultrasound diagnostic Doppler instrument, an ultrasound holographic diagnostic device, or the like, or any combination thereof. In some embodiments, the medical imaging device 110 may be configured to obtain imaging data. The imaging data may be displayed and/or recorded as a feature associated with the object in the form of a waveform, a curve, or an image. For example, the imaging data obtained by the medical imaging device 110 may include a first image and/or at least one second image. The first image and/or the at least one second image may include an ultrasound image, an MRI image, a CT image, or the like, or any combination thereof. It should be noted that the image type of the first image and the image type of the at least second image may be the same. For example, when the first image is an ultrasound image, the at least second image may also be an ultrasound image.

In some embodiments, the medical imaging device 110 may send the imaging data to the processing device 130, the terminal device 150, and/or the storage device 160 through the network 140 for further processing. For example, the medical imaging device 110 may send the at least one second image to processing device 130, and the processing device 130 may determine a target mapping relationship based on the at least one second image and thermometric data. As another example, the medical imaging device 110 may send the first image to the processing device 130, and the processing device 130 may determine a target image by performing a temperature mapping processing based on the target mapping relationship.

The infrared thermometric device 120 may be configured to perform an infrared thermometry on the object. In some embodiments, the infrared thermometric device 120 may be configured to obtain the thermometric data. The thermometric data may be displayed in the form of data or images to display a temperature distribution of the object. For example, the thermometric data may include an infrared thermal image. In some embodiments, the infrared thermometric device 120 may measure the temperature of the body surface and determine the temperature of tissue in the body based on a conversion relationship between the temperature of the body surface and the temperature of the tissue in the body. In some embodiments, the infrared thermometric device 120 may include an infrared thermal imager, an infrared thermal television, an infrared thermometer, or the like, or any combination thereof.

In some embodiments, the infrared thermometric device 120 may send the thermometric data to the processing device 130, the terminal device 150, and/or the storage device 160 through the network 140 for further processing. For example, the infrared thermometric device 120 may send the infrared thermal image to the processing device 130, and the processing device 130 may determine the target mapping relationship based on the at least one second image and the infrared thermal image.

The processing device 130 may process data and/or information that is obtained from the medical imaging device 110, the infrared thermometric device 120, the terminal device 150, and/or the storage device 160. For example, the processing device 130 may obtain the at least one second image of the object and the infrared thermal image of the object. The processing device 130 may determine a temperature curve based on a first gray value of a first pixel point corresponding to at least one first target location in the infrared thermal image. The processing device 130 may obtain the target mapping relationship based on the at least one second image and the temperature curve. As another example, the processing device 130 may obtain the first image of the object. The processing device 130 may obtain a target temperature parameter by performing a first processing on the first image based on the target mapping relationship. The processing device 130 may determine the target image based on the target temperature parameter and the first image. In some embodiments, the target image may be sent to the terminal device 150 and displayed on one or more display devices of the terminal device 150.

In some embodiments, the processing device 130 may be a single server or a server cluster. The server cluster may be centralized or distributed. In some embodiments, the processing device 130 may be local or remote. For example, the processing device 130 may access the information and/or data from the medical imaging device 110, the infrared thermometric device 120, the terminal device 150, and/or the storage device 160 through the network 140. As another example, the processing device 130 may be directly connected to the medical imaging device 110, the infrared thermometric device 120, the terminal device 150, and/or the storage device 160 to access the information and/or data. In some embodiments, the processing device 130 may be implemented on a cloud platform. For example, the cloud platform may include a private cloud, a public cloud, a hybrid cloud, a community cloud, a distributed cloud, an inter-cloud, a multi-cloud, or the like, or any combination thereof.

The network 140 may include any suitable network capable of facilitating the exchange of information and/or data of the system 100 for medical image processing. In some embodiments, information may be exchange d between one or more components (e.g., medical imaging device 110, infrared thermometric device 120, the processing device 130, the terminal device 150, the storage device 160, etc.) of the system 100 for medical image processing through network 140. For example, the processing device 130 may receive the imaging data from the medical imaging device 110 through the network 140. As another example, the processing device 130 may read data stored by the storage device 160 through the network 140.

In some embodiments, the network 140 may be any one or more of a wired network or a wireless network. For example, the network 140 may include a cable network, a fiber optic network, a telecommunications network, the Internet, a local region network (LAN), a wide region network (WAN), a wireless local region network (WLAN), a metropolitan region network (MAN), a public switched telephone network (PSTN), a Bluetooth network, a ZigBee network (ZigBee), a near field communication (NFC), an intra-device bus, an intra-device line, a cable connection, or the like, or any combination thereof. The network connection between the components may be in one of the above ways or a plurality of ways. In some embodiments, the network 140 may be in various topologies such as point-to-point, shared, centralized, or a combination thereof. In some embodiments, the network 140 may include one or more network access points. For example, the network 140 may include wired or wireless network access points, such as base stations and/or network switching points, through which one or more components of the system 100 for medical image processing may be connected to the network 140 to exchange the data and/or information.

The terminal device 150 may communicate with and/or be connected to the medical imaging device 110, the infrared thermometric device 120, the processing device 130, and/or the storage device 160. For example, the terminal device 150 may send one or more control instructions to the imaging device 110 through the network 140 to control the medical imaging device 110 to perform imaging on the object according to the instructions. In some embodiments, the terminal device 150 may include one of a mobile device 150-1, a tablet 150-2, a laptop 150-3, a desktop computer 150-4, other devices having input and/or output capabilities, or any combination thereof. In some embodiments, the terminal device 150 may include an input device, an output device, etc. The input device may include a keyboard, a touch screen, a mouse, a voice device, or the like, or any combination thereof. The output device may include a display, a speaker, a printer, or the like, or any combination thereof. In some embodiments, the terminal device 150 may be part of the processing device 130. In some embodiments, the terminal device 150 may be integrated with the processing device 130 as an operator console for the medical imaging device 110.

The storage device 160 may store the data, the instructions, and/or any other information. In some embodiments, the storage device 160 may store the imaging data that is obtained by the medical imaging device 110 and the thermometric data that is obtained by the infrared thermometric device 120. In some embodiments, the storage device 160 may store data that is obtained from the medical imaging device 110, the infrared thermometric device 120, the processing device 130, and/or the terminal device 150. For example, when the medical imaging device 110 and the infrared thermometric device 120 send obtained data to the processing device 130 for further processing, the processing device 130 may store processed data (e.g., the target mapping relationship, etc.) to the storage device 160. In some embodiments, the storage device 160 may store the data and/or the instructions that the processing device 130 uses to execute or uses to accomplish exemplary methods described herein.

In some embodiments, the storage device 160 may include a mass storage device, a removable memory, a volatile read/write memory, a read-only memory (ROM), or the like, or any combination thereof. In some embodiments, the storage device 160 may be implemented on the cloud platform.

In some embodiments, the storage device 160 may be connected to the network 140 to communicate with at least one other component (e.g., the medical imaging device 110, the infrared thermometric device 120, the processing device 130, the terminal device 150, etc.) in the system 100 for medical image processing. At least one component of the system 100 for medical image processing may access the data or the instructions stored in the storage device 160 through the network 140. In some embodiments, the storage device 160 may be part of the processing device 130.

It should be noted that the above description is merely provided for illustration purposes, and not intended to limit the scope of the present disclosure. For those skilled in the art, various amendments and variations may be made under the teachings of the present disclosure. The features, structures, methods, and other characteristics of the exemplary embodiments described in the present disclosure may be combined in various ways to obtain additional and/or alternative exemplary embodiments. For example, the medical imaging device 110, the infrared thermometric device 120, the processing device 130, and the terminal device 150 may share a common storage device 160, or may have separate storage devices. However, these amendments and variations will not depart from the scope of the present disclosure.

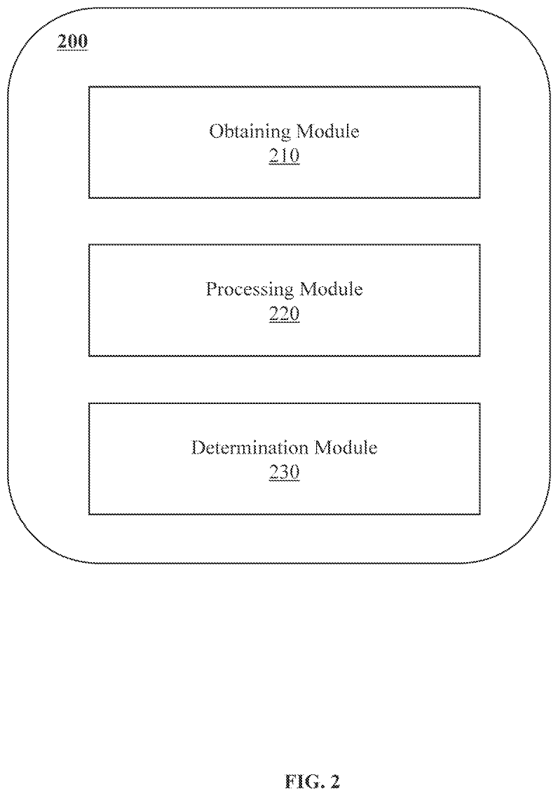

FIG. 2 is a block diagram illustrating a structure of an exemplary system for medical image processing according to some embodiments of the present description.

In some embodiments, as shown in FIG. 2, a system 200 for medical image processing may include an obtaining module 210, a processing module 220, and a determination module 230. In some embodiments, the system 200 for medical image processing may be implemented by the processing device 130. That is, the obtaining module 210, the processing module 220, and the determination module 230 may be portions of the processing device 130.

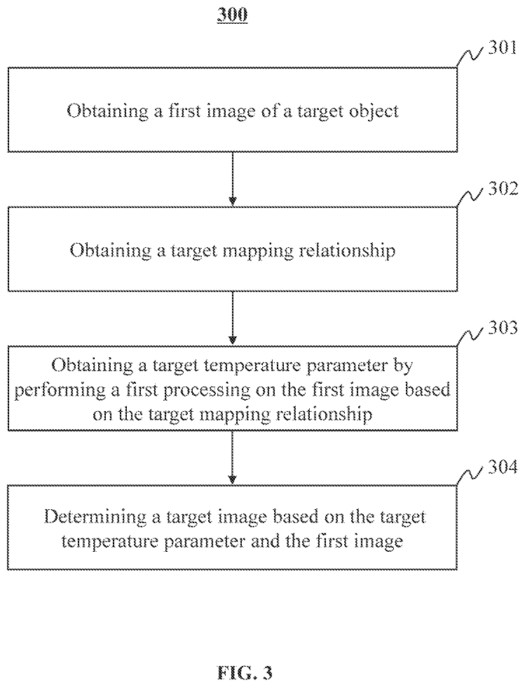

The obtaining module 210 may be configured to obtain a first image of a target object. More descriptions regarding the target object and the first image may be found in FIG. 3 and its related descriptions.

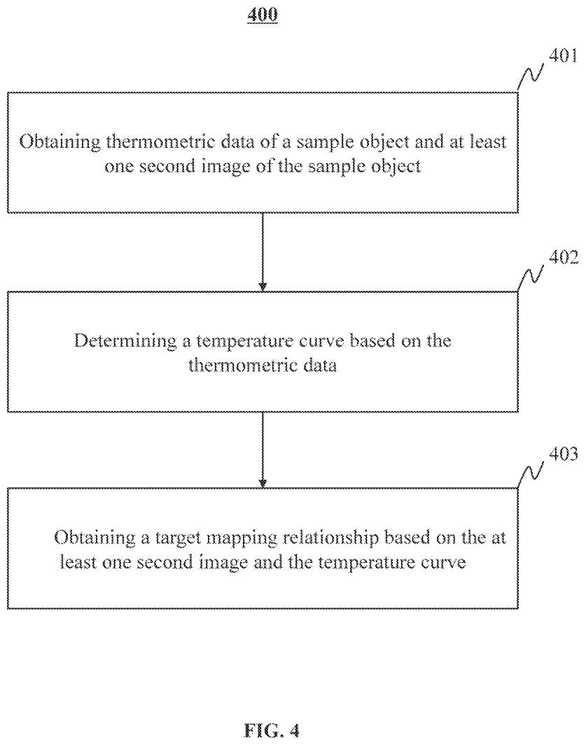

The processing module 220 may be configured to obtain a target mapping relationship, and obtain a target temperature parameter by performing a first processing on the first image based on the target mapping relationship. More descriptions regarding the first processing and the target temperature parameter may be found in FIG. 3 and its related descriptions. More descriptions of obtaining the target mapping relationship may be found in FIG. 4 and its related descriptions.

In some embodiments, to obtain the target mapping relationship, the processing module 220 may be configured to obtain thermometric data of a sample object and at least one second image of the sample object. The thermometric data may include a first temperature of at least one first target location. The processing module 220 may also be configured to determine a temperature curve based on the thermometric data, and determine the target mapping relationship based on the at least one second image and the temperature curve. In some embodiments, to determine the temperature curve based on the thermometric data, the processing module 220 may determine the temperature curve based on a first gray value of a first pixel point corresponding to the at least one first target location in the infrared thermal image. The temperature curve may include at least a correspondence between the first temperature and a second temperature corresponding to at least one second target location. In some embodiments, to obtain the target mapping relationship based on the at least one second image and the temperature curve, the processing module 220 may be further configured to determine the correspondence between a second gray value of a second pixel point corresponding to the at least one second target location in the at least one second image and the second temperature in the temperature curve, and determine the target mapping relationship by fitting the second gray value and the second temperature based on the correspondence. More descriptions of obtaining the target mapping relationship may be found in FIG. 4 and its related descriptions.

In some embodiments, when the at least one second image includes a plurality of second images, the processing module 220 may be configured to obtain a plurality of correspondence relationships by determining the correspondence between the second gray value of the second pixel point corresponding to the at least one second target location in each of the plurality of second images and the second temperature in the temperature curve; determine a plurality of candidate mapping relationships by fitting the second gray value in each of the at least one second image and the second temperature based on the plurality of correspondences; and determine the target mapping relationship by performing a second processing on a plurality of candidate mapping relationships. More descriptions regarding the plurality of candidate mapping relationships and the second processing may be found in FIG. 4 and its related descriptions.

The determination module 230 may be configured to determine a target image based on the target temperature parameter and the first image. In some embodiments, to determine the target image based on the target temperature parameter and the first image, the determination module 230 may be configured to determine the target image by performing a third processing on the first pixel point in the first image based on the target temperature parameter. More descriptions regarding the target image and the third processing may be found in FIG. 3 and its related descriptions.

It should be noted that the above descriptions are merely provided for illustration purposes, and not intended to limit the present disclosure within the scope of the embodiments. For those skilled in the art, after understanding the principle of the system, it is possible to freely combine various modules or connect sub-systems with other modules without departing from the principle. In some embodiments, the obtaining module 210, the processing module 220, and the determination module 230 disclosed in FIG. 2. may be different modules in one system, or one module that can implement functionalities of two or more of the above modules. For example, each module may share a common storage module, and each module may also have its own storage module. Such variations are within the scope of protection of the present disclosure.

FIG. 3 is a flowchart illustrating an exemplary process for medical image processing according to some embodiments of the present disclosure. In some embodiments, the process 300 for medical image processing may be performed by the system 100 for medical image processing (e.g., the processing device 130) or the system 200 for medical image processing. For example, the process 300 may be stored in a storage device (e.g., the storage device 160) in a form of programs or instructions that may implement the process 300 when the processing device 130 or the system 200 for medical image processing executes the programs or instructions. The schematic diagram that illustrates operations of the process 300 illustrated below is illustrative. In some embodiments, one or more additional operations not described and/or one or more operations not discussed may be utilized to complete the process. Further, an order of the operations of the process 300 illustrated in FIG. 3 described below is not limited.

In 301, a first image of the target object may be obtained. In some embodiments, operation 301 may be performed by the obtaining module 210.

The target object may be an object for which imaging detection is performed. In some embodiments, the target object may be an object used in a practical application stage of determining a target image based on a target mapping curve. In some embodiments, the target object may include a living organism, an artificial object, etc. In some embodiments, the target object may include a specific part of the body, e.g., the head, the neck, the chest, or the like, or any combination thereof. In some embodiments, the target object may include a specific organ, e.g., a liver, the kidney, the pancreas, the bladder, the uterus, or the like, or any combination thereof.

The first image may refer to image data obtained after imaging detection by a medical imaging device. In some embodiments, the first image may be image data obtained after imaging detection of the target object in the practical application stage.

In some embodiments, the first image may include an ultrasound image, an MR image, a CT image, or the like, or any combination thereof. The ultrasound image may be obtained by the ultrasound imaging device, the MR image may be obtained by an MRI device, and the CT image may be obtained by a CT device.

In some embodiments, the first image may be image data including grayscale information. Each pixel point (which also be referred to as a second pixel point) in the first image may correspond to one gray value (which also be referred to as a second gray value). A gray value may be used to describe a brightness or darkness level of a pixel.

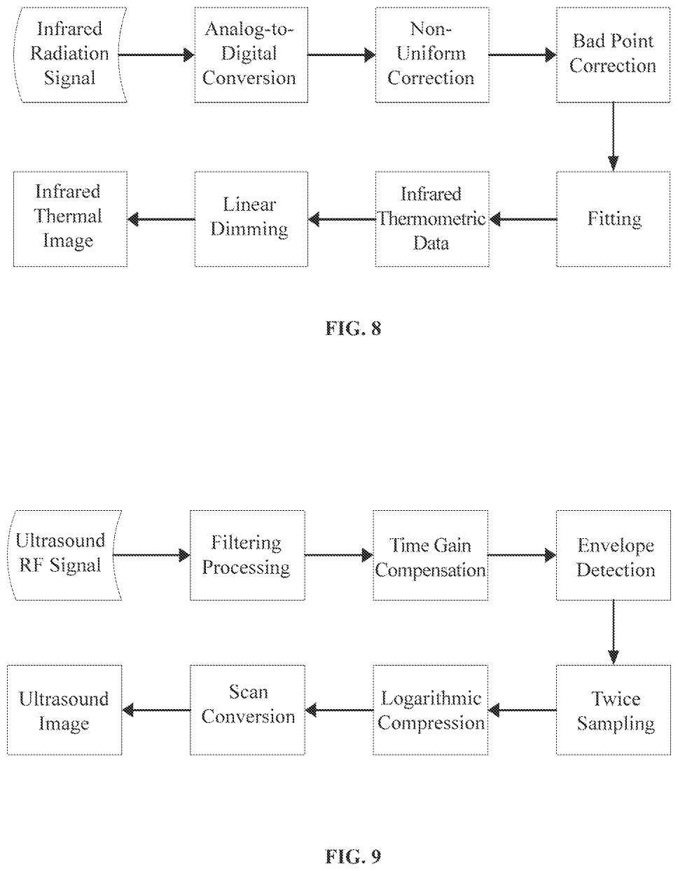

Taking ultrasound as an example, in some embodiments, the obtaining module 210 may obtain the ultrasound image of the target object by performing an image processing on ultrasound echo data (including ultrasound emission parameters and ultrasound reception parameters) of the target object collected by the ultrasound imaging device after beamforming. The image processing may include displaying received ultrasound echo data in order according to an echo strength with light and dark different photoelectricity to obtain the processing of the image data. An exemplary image processing may include a filtering processing, a time gain compensation, an envelope detection, etc. More descriptions of obtaining the ultrasound image may be found in FIG. 9 and its related descriptions.

In 302, a target mapping relationship may be obtained. In some embodiments, operation 302 may be performed by the processing module 220.

The target mapping relationship may be configured to represent a mapping relationship between the second gray value corresponding to the second pixel point in the first image and a second temperature. The second temperature may refer to a temperature value corresponding to at least one second target location. A second target location may be a specific part or a specific organ within the body of the object (including the target object and a sample object). For example, the second target location may be the liver, the heart, etc. of the object. Correspondingly, the second temperature may be the temperature of the specific part or the specific organ within the body of the subject.

In some embodiments, the target mapping relationship may be nonlinear, which can be expressed by a temperature-grayscale fitting curve. The temperature-grayscale fitting curve may include a mapping relationship between the second gray value and the second temperature. For example, the temperature-grayscale fitting curve may be expressed as m=a+bn² (n≥0), where n denotes the second gray value of the second pixel point, m denotes the second temperature, and a and b are coefficients. Assuming that the second gray value n is 20, then a corresponding second temperature may be m=a+400b.

In some embodiments, the target mapping relationship may be linear, which can be represented by a temperature-grayscale fitting line. The temperature-grayscale fitting line may include a mapping relationship between the second gray value and the second temperature. For example, the temperature-grayscale fitting straight line may be expressed as m=a′n+b′ (n≥0), where n denotes the second gray value of the second pixel point, m denotes the second temperature, and a′ and b′ are coefficients. Assuming that the second gray value n is 20, then the corresponding second temperature may be m=20a′+b′.

The target mapping relationship may be obtained through a plurality of ways. For example, the processing module 220 may determine the target mapping relationship by performing a statistical analysis on historical mapping relationships between the second temperature and the second gray value.

In some embodiments, the processing module 220 may obtain the thermometric data of the sample object and the at least one second image of the sample object, determine the temperature curve based on the thermometric data, and determine the target mapping relationship based on the at least one second image and the temperature curve. The thermometric data may include a first temperature of at least one first target location. More descriptions regarding the at least one second image, the first target location, the first temperature, the determination of the temperature curve, and the determination of the target mapping relationship may be found in FIG. 4 and its related descriptions.

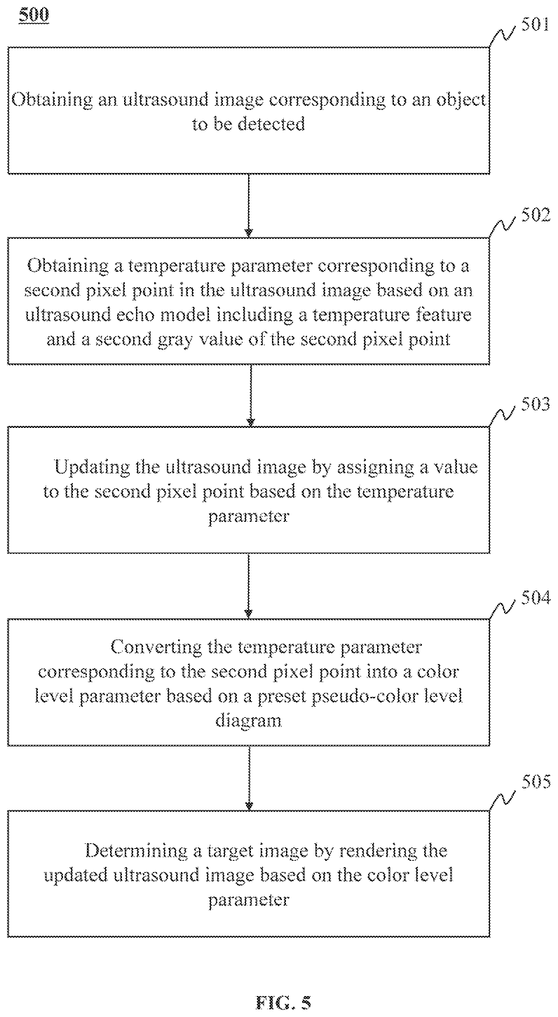

In some embodiments, the target mapping relationship may also be referred to as an ultrasound echo model including a temperature feature. More descriptions regarding the ultrasound echo model including the temperature feature may be found in FIGS. 5, 7, and 10 and their related descriptions.

In 303, a target temperature parameter may be obtained by performing a first processing on the first image based on the target mapping relationship. In some embodiments, operation 303 may be performed by the processing module 220.

In some embodiments, the performing the first processing on the first image may refer to performing a temperature mapping processing on the first image. The temperature mapping processing may refer to a processing that maps a grayscale set to a temperature set based on a determined correspondence (i.e., the target mapping relationship). For example, the temperature mapping processing may be a processing that determines a second temperature corresponding to the second gray value in the target mapping relationship based on the second gray value corresponding to the second pixel point in the first image.

The target temperature parameter (which also be referred to as a temperature parameter) may refer to a set of temperature values used to assign values to a plurality of second pixel points in the first image. The target temperature parameter may include one or more sub-temperature parameters, each of which corresponds to one of the plurality of second pixel points. For example, the target temperature parameter may be expressed as ([1, 35.2° C.], [2, 36.3° C.], [3, 30.2° C.] . . . ), indicating that the sub-temperature parameter corresponding to a second pixel point 1 is 35.2° C., the sub-temperature parameter corresponding to a second pixel point 2 is 36.3° C., and the sub-temperature parameter corresponding to a second pixel point 3 is 30.2° C., etc.

In some embodiments, the target temperature parameter may be an actual temperature value corresponding to the at least one second target location (i.e., the second temperature), and the target temperature parameter may be configured to reflect an actual temperature value corresponding to the at least one second target location in the first image. For example, when the second target location is the kidney, the target temperature parameter may reflect actual temperature values at various locations in the kidney. In some embodiments, the target temperature parameter may also be a relative temperature value corresponding to the at least one second target location. The relative temperature value may refer to a relative value of the actual temperature of the second target location in the target object relative to the normal temperature of the second target location. For example, when the second target location is the kidney, the target temperature parameter may reflect the relative temperature value of various locations in the kidney.

In some embodiments, the processing module 220 may determine the second temperature corresponding to each second pixel point by performing the temperature mapping processing on the second gray value of each second pixel point in the first image based on the target mapping relationship, and obtain the target temperature parameter based on the second temperature. For example, when the second gray value of the second pixel point 1 in the first image is 120, the second temperature corresponding to the second gray value of 120 may be determined as 40° C. after being performed the temperature mapping processing based on the target mapping relationship, and a target thermometric temperature parameter may be determined as 40° C. or 2° C., where 2° C. is the relative temperature value. The relative temperature value may be obtained based on the second temperature corresponding to the second pixel point 1 (i.e., 40° C.) and the second pixel point 1 corresponding to the normal temperature (38° C.).

In 304, the target image may be determined based on the target temperature parameter and the first image. In some embodiments, operation 303 may be performed by the determination module 230.

In some embodiments, the target image may refer to a first image including the temperature data.

In some embodiments, the determination module 230 may display the temperature data of the target object on the basis of displaying the imaging data of the first image to obtain the target image. The temperature data may include the actual temperature value or the relative temperature value of the at least one second target location. For example, when the first image is an ultrasound image, the target image may be an image that displays both the ultrasound data and the temperature data. When the first image is an MR image, the target image may be an image that displays both the MR data and the temperature data. When the first image is a CT image, the target image may be an image that displays both the CT data and the temperature data. The image type of the target image and the image type of the first image may be the same. For example, when the first image is an ultrasound image, the target image may be an ultrasound image including the temperature data.

In some embodiments, the determination module 230 may determine the target image by performing a third processing on the second pixel point in the first image based on the target temperature parameter.

In some embodiments, the third processing may refer to a process that assigns a value to the second pixel point in the first image based on the target temperature parameter. The target temperature parameter may be overlaid and displayed on the second pixel point by assigning the value. The second pixel point after the assignment process may simultaneously display both the imaging data and the temperature data.

The manner of performing the third processing may include a plurality of ways. For example, the target temperature parameter may be converted to a text label including the temperature value or other feasible form to assign the value to the second pixel point, such that the target temperature parameter is overlaid and displayed on the second pixel point.

In some embodiments, the performing the third processing may further include converting the target temperature parameter to a color level parameter based on a preset pseudo-color level diagram, and rendering the second pixel point in the first image based on the color level parameter.

The preset pseudo-color level diagram may include the correspondence between different colors and different temperature ranges. For example, the correspondence between the different colors included in the preset pseudo-color level diagram and the different temperature ranges may be that: dark blue indicates a temperature range of 30° C.-32° C., light blue indicates a temperature range of 32° C.-34° C., yellow indicates a temperature range of 34° C.-36° C., orange indicates a temperature range of 36° C.-38° C., etc. As another example, the correspondence between the different colors included in the preset pseudo-color level diagram and the different temperature ranges may be that: dark blue indicates a temperature range of 0° C.-1° C., light blue indicates a temperature range of 1° C.-2° C., yellow indicates a temperature range of 3° C.-4° C., orange indicates a temperature range of 4° C.-5° C., etc.

The preset pseudo-color level diagram may be obtained in a plurality of ways. For example, the preset pseudo-color level diagram may be obtained by human or system presets.

In some embodiments, the color level parameter may be configured to color render the plurality of second pixel points in the first image. In some embodiments, the color level parameter may include one or more sub-color level parameters, and each sub-color level parameter may be configured to perform a color rendering on one of the plurality of second pixel points. For example, the color level parameter may be expressed as ([1, (R255, G255, B0)], [2, (R22, G07, B201)], [3, (R32, G11, B225)] . . . ), indicating that the sub-color level parameter for the color rendering of the second pixel point 1 is expressed as (R255, G255, B0), the sub-color level parameter for the color rendering of the second pixel point 2 is expressed as (R22, G07, B201), and the sub-color level parameter for the color rendering of the second pixel point 3 is expressed as (R32, G11, B225).

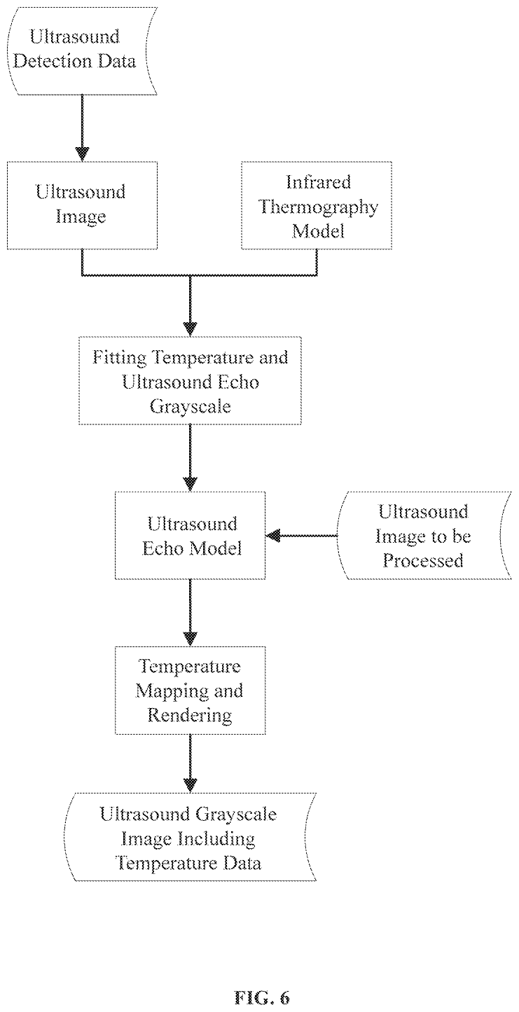

In some embodiments, the determination module 230 may determine a corresponding color level parameter for each temperature value in the target temperature parameter based on a conversion relationship between the color and the color level parameter according to a temperature range and the color of the target temperature parameter in the preset pseudo-color level diagram. For example, if the target temperature parameter of the second pixel 1 is 36.7° C., and the corresponding temperature range and its color in the preset pseudo-color level diagram may be “36° C.-38° C., orange”, then the sub-color level parameter of the second pixel 1 may be determined as (R22, G07, B201) based on the conversion relationship between the color and the color level parameter. More descriptions of determining the target image may be found in FIG. 5 and FIG. 6 and their related descriptions.

In some embodiments of the present disclosure, the target image may be obtained by assigning the target temperature parameter to the second pixel point in the first image, which allows the target image to display the temperature data of the target object on the basis of displaying the imaging data.

In some embodiments, the determination module 230 may obtain the temperature image based on the target temperature parameter, and determine the target image based on the temperature image and the first image.

The temperature image may refer to the first image including the temperature data. In some embodiments, the temperature image may include the temperature data of the at least one second target location. For example, the temperature image may include the actual temperature value of the at least one second target location. As another example, the temperature image may include the relative temperature value of the at least one second target location.

In some embodiments, the determination module 230 may determine the temperature image by performing the third processing on the second pixel point in the first image based on the target temperature parameter. The manner of performing the third processing may be as previously described, which is not repeated herein.

In some embodiments, in determining the target image based on the temperature image and the first image, the temperature image and the first image may be determined together as the target image. That is, the target image may be composed of two images. The target image may include the first image before overlaying the temperature data and the first image after overlaying the temperature data (i.e., the temperature image as above described).

In some embodiments of the present disclosure, temperature information may be introduced on the basis of traditional ultrasound detection, MR detection, or CT detection, and the imaging data including the temperature data of the target object may be obtained through an organic combination of the infrared thermometric data and the imaging data, which brings into play advantages of the ultrasound detection, the MR detection or the CT detection and also makes full use of a reference role of the infrared thermometry for the determination of target object lesions. In this way, the accuracy of a medical diagnosis may be improved by combining the temperature data on the basis of a traditional lesion or lesion tissue determination, especially providing a supportive analysis basis for medical diagnosis of very small or ambiguous lesions and pathological tissues. At the same time, by respectively displaying the imaging data before overlaying the temperature data and the imaging data after overlaying the temperature data, it may further assist doctors in diagnosis and effectively improve the efficiency of diagnosis.

In some embodiments, the determination module 230 may input the target image into a determination model to determine an abnormal region in the target image.

The determination model may be a machine learning model configured to identify an abnormal region. For example, the determination model may include a Neural Networks (NN) model, a Convolutional Neural Networks (CNN) model, a Deep Neural Networks (DNN) model, or the like, or any combination thereof.

In some embodiments, an input of the determination model may include the target image, and an output of the determination model may include the target image with the abnormal region that is labeled. The abnormal region may refer to a region of the target object where the temperature is outside a normal temperature range (e.g., a normal temperature range when the target object does not have a lesion, etc.).

In some embodiments, the determination model may label the abnormal region in the target image through an identification frame, and the output of the determination model may be the target image labeled with the identification frame. An exemplary recognition frame may include a geometric border of rectangular, circular, or other shapes.

In some embodiments, the determination model may also intercept an image (also referred to as an abnormal image) corresponding to the abnormal region and output the abnormal image after the image processing. The exemplary image processing may include, but not limited to, image magnification, image enhancement, image region segmentation, image geometric transformation, or the like, or any combination thereof.

In some embodiments, the abnormal image may include an abnormal image corresponding to the abnormal region in the first image before overlaying the temperature data and/or an abnormal image corresponding to the abnormal region in the first image after decomposing the temperature data. For example, when the input of the determination model is the first image after overlaying the temperature data, the output of the determination model may be the first image after overlaying the temperature data with the labeled abnormal region, or the output of the determination model may be the abnormal image corresponding to the abnormal region in the first image after overlaying the temperature data. When the input of the determination model is the first image before overlaying the temperature data and the first image after overlaying the temperature data, the output of the determination model may be the first image before overlaying the temperature data with the labeled abnormal region and the first image after overlaying the temperature data with the labeled abnormal region, or the output of the determination model may be the abnormal image corresponding to the abnormal region in the first image before overlaying the temperature data and the abnormal image corresponding to the abnormal region in the first image after overlaying the temperature data.

In some embodiments, the determination model may be obtained by training a plurality of training samples with labels. For example, the plurality of training samples with labels may be input into an initial determination model. A loss function may be constructed by the labels and results of the initial determination model. Parameters of the initial determination model may be updated iteratively based on the loss function. When the loss function of the initial determination model satisfies a preset condition, the model training may be completed to obtain a trained determination model. The preset condition may include a convergence of the loss function, a count of iterations reaching a preset number, etc. In some embodiments, a training sample may include a sample target image. The sample target image may include a target image having an abnormal region and a target image without the abnormal region. The label may be the abnormal region in the sample target image. The training sample may be obtained based on historical data, and the labels may be obtained by manual labeling.

In some embodiments of the present disclosure, when the temperature difference is not obvious from the normal tissue performance, it is more difficult for a doctor to identify the abnormal region by the naked eye for further diagnosis of the condition of a lesion. However, through model identification, the abnormal region having a temperature anomaly may be quickly and accurately identified, which is conducive for the doctor to make a further diagnose and effectively improves diagnostic efficiency.

In some embodiments, when the first image is the ultrasound image or the CT image, the system 100 for medical image processing may obtain an MR image using an MRI device, and process the MR image after sending the MR image to the processing device 130. The processing device 130 may further fuse the MR image with the target image to obtain a fused image (also referred to as a first fused image). The target image may be the ultrasound image or the CT image with overlaid temperature data. For example, the processing device 130 may further fuse the MR image with the first image after overlaying the temperature data to obtain the first fusion image. As another example, the processing device 130 may fuse the MR image with the abnormal image to obtain the first fusion image.

The first fusion image may be determined in a plurality of ways. For example, the MR image may be aligned with the target image by marking features or identifiable physical space locations, and an aligned MR image may be fused with the target image to obtain the first fusion image.

In some embodiments, when the first image is the ultrasound image or the MR image, the system 100 for ultrasound image processing may obtain a CT image by using a CT imaging device, and process the CT image after sending the CT image to the processing device 130. The processing device 130 may further fuse the CT image with the target image to obtain the fused image (also referred to as a second fused image). The target image may be the ultrasound image or MR image with overlaid the temperature data. For example, the processing device 130 may further fuse the CT image with the first image after overlaying the temperature data to obtain the second fused image. As another example, the processing device 130 may fuse the CT image with the abnormal image to obtain a second fused image.

The second fused image may be determined in a plurality of ways. For example, magnetic field coordinates and CT image coordinates of a plurality of reference points may be obtained by an electromagnetic positioning system. A magnetic field coordinate system and a CT image coordinate system may be aligned by using an alignment algorithm. A coordinate system where an ultrasound probe is located may be aligned with the magnetic field coordinate system using the electromagnetic positioning system. The ultrasound coordinate system may be aligned with a coordinate system of an electromagnetic sensor by using the ultrasound probe. Finally, the ultrasound coordinate system may be aligned to the CT image coordinate system through conversion relationships of a plurality of coordinate systems, and a real-time ultrasound image may be fused with the CT image to obtain the second fusion image.

In some embodiments of the present disclosure, when the lesion is difficult to detect, by introducing the temperature information on the basis of the traditional ultrasound detection, and further introducing the MR detection information or CT detection information, the ultrasound image after overlaying the temperature data may be further organically combined with the MR image or the CT image to obtain the fused image including the temperature data, ultrasound detection data and MR detection data or CT detection data of the target object, which can further be used to analyze and determine the lesion more accurately. At the same time, the ultrasound-MR image fusion technology combines the advantages of ultrasound and MRI, which can clearly display a multi-level MRI cross-sectional image associated with the ultrasound image in real time and dynamically. In addition, the ultrasound-CT image fusion technology combines the advantages of ultrasound and CT, which can clearly display the CT image associated with the ultrasound image in real time and dynamically, thereby providing technical support for the accuracy of interventional treatment.

FIG. 4 is a flowchart of an exemplary process for obtaining a target mapping relationship according to some embodiments of the present disclosure. In some embodiments, the process for obtaining the target mapping relationship may be performed by the system 100 for medical image processing (e.g., the processing device 130) or the system 200 for medical image processing (e.g., processing module 220). For example, the process 300 may be stored in the storage device (e.g., storage device 160) in the form of the program or the instructions, and when the processing device 130 or the system 200 for medical image processing executes the program or instructions, the process 400 may be implemented. The schematic diagram illustrating operations of the process 400 presented below is illustrative. In some embodiments, one or more additional operations that are not described and/or one or more operations that are not discussed may be utilized to complete the process. Further, the order of the operations of the process 400 illustrated in FIG. 4 described below is not limited.

In 401, thermometric data of a sample object and at least one second image of the sample object may be obtained.

The sample object may be an object on which imaging detection is performed. In some embodiments, the sample object may be the object used in a pre-processing stage configured to obtain a target mapping curve. Similar to the target object, the sample object may be a living organism, an artificial object, a specific part or a specific organ of the body, etc. In some embodiments, the target object may also be an object used in the pre-processing stage of the target mapping curve and configured to obtain the target mapping curve. That is, the target object may be the same object as the sample object.

The thermometric data may refer to temperature data associated with at least one first target location of the sample subject. The first target location may be a specific part of the body surface of the sample subject, etc. For example, the first target location may be the armpit, the forehead, the palm, etc. of the sample subject. It is noted that there is an association relationship between each of the at least one first target location and a certain second target location of at least one second target location. For example, the association relationship between a certain first target location and a certain second target location may be that the second target location is a same location point or location region at a subcutaneous depth of 10 cm of the first target location.

In some embodiments, the thermometric data may include infrared signal data reflecting a first temperature of the at least one first target location. The infrared signal data (also referred to as an infrared radiation signal) may be obtained by a thermometric device. The first temperature may refer to temperature data of the body surface of the object. For example, the infrared signal data of the at least one first target location may be obtained by the infrared thermal imager, the infrared thermal television, the infrared thermometer, or the like, or any combination thereof.

In some embodiments, the processing module 220 may obtain a first temperature of the at least one first target location by receiving the infrared radiation signal detected by the thermometric device and performing an analog-to-digital conversion, a non-uniform correction, a bad point correction, a fitting, or other processing.

In some embodiments, the thermometric data may also be corrected to reduce bias in a collection of the thermometric data caused by environmental temperature differences, individual differences, etc. For example, a correction model may be trained based on environmental information (e.g., summer, winter, daytime, nighttime, room temperature, etc.), individual difference information (e.g., male, female, child, etc.), and medical pathology information (e.g., whether there is a problem in the liver to be examined, whether the patient has other medical conditions associated with the liver lesion, or whether there are other conditions around the liver that may affect the liver temperature), and configured to calibrate the thermometric data based on trained correction model. In some embodiments, the correction model may include a Recurrent Neural Network (RNN) model, a Deep Neural Network (DNN) model, a Convolutional Neural Network (CNN) model, or the like, or any combination thereof.

In some embodiments, the thermometric data may include an infrared thermal image. In some embodiments, the infrared thermal image may be image data including grayscale information. Each pixel point (which may become a first pixel point) in the infrared thermal image may correspond to one gray value (also referred to as a first gray value). In some embodiments, the processing module 220 may obtain the infrared thermal image by receiving the infrared radiation signal detected by the infrared thermometric device and performing a processing such as the analog-to-digital conversion, the non-uniform correction, the bad point correction, the fitting, the linear dimming, etc. More descriptions of obtaining the infrared thermal image may be found in FIG. 8 and its related descriptions.

The at least one second image may refer to the image data that is obtained after imaging detection by the medical imaging device. In some embodiments, the at least one second image may be image data that is obtained after imaging detection of the sample object in the pre-processing stage. In some embodiments, the at least one second image may be image data that is obtained after imaging detection of the specific part or the specific organ by the medical imaging device.

In some embodiments, the at least one second image may include the ultrasound image, the MRI image, the CT image, or the like, or any combination thereof. The image type of the at least one second image and the image type of the first image may be same.

In some embodiments, the at least one second image may be image data including grayscale information. Each pixel point (also referred to as the first pixel point) in the at least one second image may correspond to one gray value (also referred to as a first gray value).

The first image and the at least one second image may be obtained in a similar way, with the difference that obtaining stages of the first image and the at least one second image are different. The at least one second image may be the image data that is obtained by the imaging detection of the sample object in the pre-processing stage of obtaining the target mapping relationship. The first image may be the image data that is obtained by the imaging detection of the target object in a practical application stage of applying the target mapping curve to determine the target image. More descriptions of obtaining the first image or the at least one second image may be found in FIG. 9 and its related descriptions.

In 402, a temperature curve may be determined based on the thermometric data.

The temperature curve (also referred to as an infrared thermometric model) may refer to a curve associated with a temperature value.

In some embodiments, the temperature curve may be determined by constructing a first mapping equation based on the thermometric data. An exemplary first mapping equation may be: k=qi²+pi+v, i.e., the temperature curve satisfies part of the needs of a parabolic mathematical definition, where k denotes a dependent variable, i denotes an independent variable, and q, p, and v are constants.

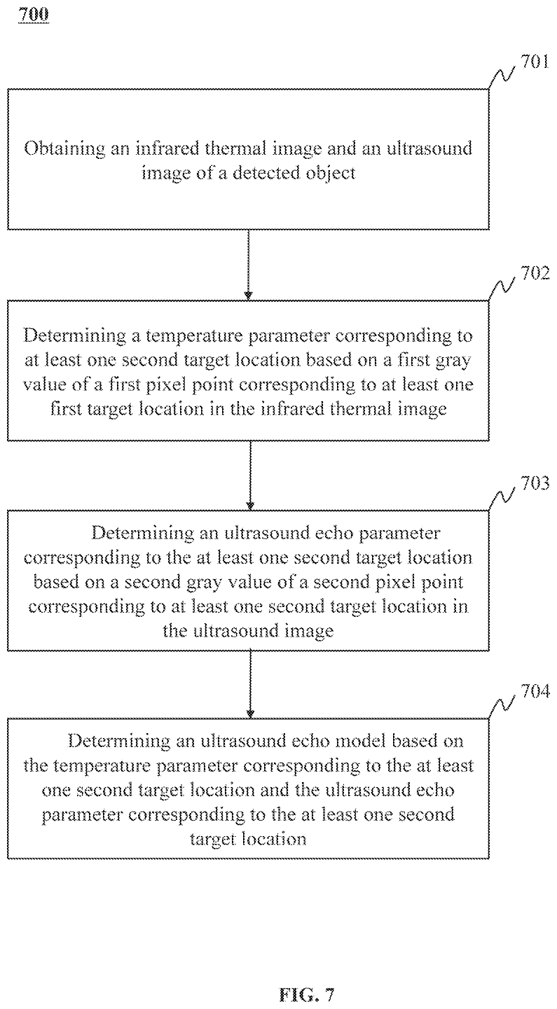

In some embodiments, the temperature curve may include the correspondence between the infrared signal data of the at least one first target location and the first temperature corresponding to the at least one first target location. A horizontal axis of the temperature curve may represent the infrared signal data of the at least one first target location, and a vertical axis of the temperature curve may represent the first temperature of the at least one first target location. Accordingly, the construction of the first mapping relationship equation may include: substituting infrared signal data of each of the at least one first target location and the first temperature into the first mapping equation to determine the temperature curve, where k denotes the first temperature, and i denotes the infrared signal data. More descriptions regarding the above embodiments may be found in FIG. 7 and its related descriptions.

In some embodiments, the temperature curve may include the correspondence between the infrared signal data of the at least one first target location and the second temperature corresponding to the at least one second target location. The horizontal axis of the temperature curve may represent the infrared signal data of the at least one first target location, and the vertical axis of the temperature curve may represent the second temperature of the at least one second target location. Accordingly, the construction of the first mapping relationship equation may include: substituting infrared signal data of each of the at least one first target location and the second temperature of the at least one second target location into this first mapping equation to determine the temperature curve, where k denotes the second temperature and i denotes the infrared signal data.

In some embodiments, the temperature curve may include the correspondence between the first temperature of the at least one first target location and the second temperature of the at least one second target location. The horizontal axis of the temperature curve may represent the first temperature of the at least one first target location, and the vertical axis of the temperature curve may represent the second temperature of the at least one second target location. Accordingly, the construction of the first mapping equation may include: substituting a plurality of first temperatures at a first target location and a plurality of second temperatures at a second target location into the first mapping equation to determine the temperature curve, where k denotes the second temperature and i denotes the first temperature.

The second temperature may be determined in a plurality of ways. For example, the second temperature may be determined by measuring tissues at different depths in the body of the sample subject through a device such as a probe thermometer, a magnetic resonance imaging thermometer, or a fiber optic thermometric device. As another example, the second temperature may be determined according to the first temperature through the temperature conversion relationship between the first temperature and the second temperature. As an example, when an axillary temperature of the human body is measured to be 36° C., the temperature at a location of an axillary subcutaneous of 2 cm may be determined as 36.2° C.-36.4° C., etc. according to the temperature conversion relationship between the axilla and the location of the axillary subcutaneous of 2 cm (an exemplary conversion relationship is that: the temperature of the location of the axillary subcutaneous of 2 cm may be 0.2° C.-0.4° C. higher than the axillary temperature).

In some embodiments, the temperature curve may include the correspondence between the first gray value corresponding to the first pixel point in the infrared thermal image and the first temperature corresponding to the at least one first target location. The horizontal axis of the temperature curve may represent the first gray value corresponding to first pixel point in the infrared thermal image, and the vertical axis of the temperature curve may be the first temperature corresponding to the at least one first target location. In some embodiments, the temperature curve may be determined based on the first gray value of the at least one first target location corresponding to the first pixel point in the infrared thermal image. Accordingly, the construction of the first mapping equation may include substituting first gray values of a plurality of first pixel points in the infrared thermal image with first temperatures corresponding to the first pixel points into the first mapping equation and determining the temperature curve based on the mapping equation, where k denotes the first gray value and i denotes the first temperature.

In some embodiments, the temperature curve may include the correspondence between a first gray value corresponding to the first pixel point in the infrared thermal image and a second temperature corresponding to the at least one second target location. The horizontal axis of the temperature curve may represent the first gray value corresponding to the first pixel point in the infrared thermal image, and the vertical axis of the temperature curve may represent the second temperature corresponding to the at least one second target location. In some embodiments, the temperature curve may be determined based on the first gray value of the at least one first target location corresponding to the first pixel point in the infrared thermal image. Accordingly, the construction of the first mapping equation includes: substituting first gray values of the plurality of first pixel points in the infrared thermal image with the second temperatures corresponding to the first pixel points into the first mapping equation and determining the temperature curve based on that mapping equation, where k denotes the second temperature and i denotes the first gray value. The correspondence between the first pixel point and the second temperature may be determined based on the conversion relationship between the first pixel point and the first temperature, and the conversion relationship between the first temperature and the second temperature. For example, the first temperature corresponding to a first pixel point A may be 36° C. A first target location where the pixel point A is located may be the axilla, and a second target location where the second temperature is located may be a location of the axillary subcutaneous of 2 cm. Then, the second temperature may be determined as 36.2° C.-36.4° C., and finally, a second temperature corresponding to the first pixel point A may be obtained as 36.2° C.-36.4° C.

In 403, a target mapping relationship may be obtained based on the at least one second image and the temperature curve.

In some embodiments, the processing module 220 may determine a correspondence between the second gray value of the second pixel point corresponding to at least one second target location in the at least one second image and the second temperature in the temperature curve, and determine the target mapping relationship by fitting the second gray value to the second temperature based on the correspondence.

In some embodiments, the correspondence between the second grayscale value of a second pixel point and the second temperature in the temperature curve may refer to a correspondence management that associates the second grayscale value of a specific second pixel point with a specific second temperature. For example, if the second gray value corresponding to the second pixel point A is 120, the second target location where the second pixel point A is located is B, and the second temperature of the second target location B is 36.7° C., then the correspondence may be obtained by associating the second gray value of 120 with the second temperature of 36.7° C.

In some embodiments, the processing module 220 may fit the plurality of second gray values and the plurality of second temperatures based on the correspondence between the second gray value of the second pixel point and the second temperature in the temperature curve. Though the fitting, each of the second gray values of the plurality of second gray values may be associated with one of the second temperatures of the plurality of second temperatures. In some embodiments, the manner of determining the target mapping relationship may include, but not limited to, constructing a second mapping equation. The independent variable of the second mapping equation may be the second gray value of a second pixel point and the dependent variable may be a second temperature. An exemplary second mapping equation may be expressed as y=a₀+a₁x+a₂x²+ . . . +aₖx^{k}, where x denotes the second gray value of the second pixel point, y denotes the second temperature, and the equation coefficients a₀, a₁, a₂, aₖ are determined by performing a fitting processing on multiple calibrated sets (x, y), and the fitting processing includes, but not limited to, a least squares fitting, an interpolation approximation fitting, etc. The multiple calibrated sets (x, y) may be determined based on the correspondence between the second gray value of the second pixel point and the second temperature in the temperature curve.

In some embodiments, when the at least one second image includes the plurality of second images, the processing module 220 may obtain a plurality of correspondences by determining the correspondence between the second gray value of the second pixel point corresponding to at least one second target location in each of the at least one second image and the second temperature in the temperature curve. The processing module 220 may determine a plurality of candidate mapping relationships by fitting the second gray value of each of the at least one second image and the second temperature based on the plurality of correspondence relationships. The processing module 220 may determine the target mapping relationship by performing second processing on the plurality of candidate mapping relations. It is noted that the plurality of second images may be a plurality of images of the same specific part or the specific organ.

In some embodiments, the plurality of second images may be obtained by performing a plurality of imaging detections on the same specific part or specific organ of the sample object.

The manner of determining each candidate mapping relation may be the same as the above manner of determining the target mapping relation, which is not repeated herein.

In some embodiments, the second processing may refer to a processing that obtains a final target mapping relationship by fusing the plurality of candidate mapping relationships. In some embodiments, the manner of performing the second processing may include, but not limited to, a linear fitting, an equation fitting, a least squares fitting, etc. The final target mapping relationship may be obtained through the second processing. For example, when the plurality of candidate mapping relationships are a plurality of temperature-grayscale fitting curves or a plurality of temperature-grayscale fitting straight lines, the plurality of temperature-grayscale fitting curves or the plurality of temperature-grayscale fitting straight lines may be fitted by the least squares fitting as a single temperature-grayscale fitting curve or a single temperature-grayscale fitting straight line to obtain the target mapping relationship.

It is noted that when the plurality of second images are from the same specific part or specific organ (which may be from the same specific part or specific organ of the same or different sample objects), the correspondence between the second gray value of the second pixel point corresponding to the at least one second target location in each of the at least one second image and the second temperature in the temperature curve may be the same or different. The target mapping relationship corresponding to the specific part or the specific organ may be obtained by fitting the correspondence between the second gray value of the second pixel point corresponding to at least one second target location in each of the at least one second image of the specific part or the specific organ and the second temperature in the temperature curve.

In some embodiments of the present disclosure, by obtaining the target mapping relationship, the infrared thermal image may be achieved to be organically combined with the ultrasound echo image, the MR image or the CT image, which gives full play to the advantages of the ultrasound echo data detection, the MR detection or the CT detection while also making full use of the reference role of the infrared thermometry for the lesion determination, and provides the auxiliary determination by combining the relative temperature information on the basis of the traditional lesion or pathological tissue determination, thereby improving the accuracy of the medical diagnosis, specifically providing a reliable analysis for the medical diagnosis of the very small or ambiguous lesions and the pathological tissues. Meanwhile, the plurality of second images may be obtained by performing ultrasound imaging, MR imaging or CT imaging on different specific parts or specific organs of the human body at one time, and the plurality of candidate mapping relationships may be obtained by determining the correspondence between the second gray value of the second pixel point in each of the at least one second image and the second temperature in the temperature curve, and a more accurate and applicable target mapping relationship for a plurality of specific parts or specific organs of the human body may be determined by fitting the plurality of candidate mapping relationships. Furthermore, the plurality of second images may be obtained by performing a plurality of ultrasound imagings, MR imagings, or CT imagings of the specific part or specific organ of the human body. The plurality of candidate correspondences may be obtained by determining a correspondence between the second grayscale value of the second pixel point in each of the at least one second image and the second temperature in the temperature curve. A more accurate target mapping relationship for the specific part or specific organ of the human body may be determined by fitting the plurality of candidate mapping relationships.

It should be noted that the above description of the process 300 and the process 400 are merely provided for illustration purposes, and not intended to limit the scope of the present disclosure. For those skilled in the art, various amendments and variations may be made to the process 300 and the process 400 under the teachings of the present disclosure. However, these amendments and variations remain within the scope of the present disclosure.

FIG. 7 is a flowchart illustrating an exemplary process for obtaining an ultrasound echo model including a temperature feature according to some embodiments of the present disclosure. Referring to FIG. 7, the present embodiment specifically provides the process for obtaining an ultrasound echo model including a temperature feature, including the following operations.

In 701, an infrared thermal image of a detected object and an ultrasound image of a the detected object may be obtained.

In 702, a temperature parameter corresponding to at least one second target location may be determined based on a first gray value of a first pixel point corresponding to at least one first target location in the infrared thermal image.

In 703, an ultrasound echo parameter corresponding to at least one second target location may be determined based on a second gray value of a second pixel point corresponding to the at least one second target location in the ultrasound image.

In 704, the ultrasound echo model may be determined based on the temperature parameter corresponding to the at least one second target location and the ultrasound echo parameter corresponding to the at least one second target location. The ultrasound echo model may be configured to map the ultrasound echo parameters of different second target locations of detected objects to the temperature parameters.

As described in 701, the infrared thermal image and the ultrasound image of the detected object may be obtained. The detected object may include, but not limited to, human and animal body tissues, and the present embodiment is illustrated by taking the human body tissues as an example. It should be noted that the detected object may be other objects different from the object to be detected, or the same object as the object to be detected.

In some embodiments, the temperature parameters may be obtained by performing image analysis on the infrared thermal image. An object above absolute zero constantly radiates and absorbs infrared radiation, and the infrared radiation is closely related to human blood circulation, tissue metabolism, and neural functional state. The human body, as a thermogenic organism, has different dimensions in various parts, and a temperature distribution of a normal body exhibits a certain degree of stability and symmetry. During a metabolic process, cells of the detected object generate heat and transmit the heat to the body surface in the form of thermal radiation, and heat from deep tissues can be transferred to the body surface through blood flow and inter-tissue conduction. Therefore, a presence of a lesion in a certain part of the human body often affects the temperature stability of the detected object in the region. The first gray value of the first pixel point in the infrared thermal image may be determined based on the infrared thermometric model corresponding to the detected object and the infrared thermometric data corresponding to the detected object. The infrared thermometric data may be the independent variable of the infrared thermometric model, and the dependent variable of the infrared thermometric model may be the first gray value of the first pixel point. In some embodiments, the present disclosure may analyze the temperature variation at the lesion by analyzing the state of the body temperature distribution displayed by the infrared thermometric data, thereby making a preliminary determination of the lesion. The present disclosure may also use infrared thermometric technology to partially convert an object surface temperature into an image visible to the human eye, and display a surface temperature distribution of the object in different colors. A basic process of obtaining the infrared thermal image may include using an infrared lens to receive and converge the infrared radiation emitted by the detected object. The thermal radiation signals may then be converted into electrical signals through the device such as an infrared detector, using the analog-to-digital conversion. The electrical signals may be further processed through associated electronic components for a non-uniformity correction, a bad pixel correction, etc. The temperature curve may be fitted based on the infrared thermometric model, such that the infrared thermal image may be obtained by combining the infrared temperature data and performing a linear dimming and the like. The basic process may be illustrated in FIG. 8. It is known to those skilled in that art, the above intermediate processing does not constitute a limitation to the present disclosure.

Ins some embodiments, the infrared thermometric model may indicate a curve correspondence relationship between the original infrared thermometric data and the human body temperature. The horizontal axis expresses the infrared signal data and the vertical axis expresses the human body temperature data. The human body temperature data may be the gray value of the pixel point in the infrared thermal image. In some embodiments, the mapping equation may be: y=ax²+bx+c, that is, the curve of the infrared thermometric model satisfies part of the needs of a parabolic mathematical definition, where a, b, and c are constants.

The ultrasound echo parameter may be obtained through an analysis of the ultrasound image. In some embodiments, ultrasound imaging technology is an examination manner that uses the physical properties of ultrasound and differences in acoustic properties of human organs and tissues and is displayed and recorded in the form of waveforms, curves, or images for disease diagnosis. Various organs and tissues in the human body have their specific acoustic impedances and attenuation characteristics, which constitute differences in acoustic impedance and attenuation. The ultrasound emitted into the body from surface to depth would pass through organs and tissues with different acoustic impedances and different attenuation characteristics, thereby generating different reflections and attenuations. The reflections and attenuations may be utilized to form an ultrasound image.

As described in 702, the temperature parameter corresponding to the at least one second target location may be determined based on the first gray value of the first pixel point corresponding to the at least one first target location in the infrared thermal image.

As described in 703, the ultrasound echo parameter corresponding to the second target location may be obtained based on the second gray value of the second pixel point corresponding to the second target location in the ultrasound image corresponding to the detected object. The first target location and the second target location may be determined according to a need for lesion analysis of the body tissue, a need for post-fitting processing, or other needs. In some embodiments, the ultrasound echo parameter may be obtained by beamforming based on an ultrasound emission parameter of the second target location and an ultrasound reception parameter. In some embodiments, received echoes may be displayed on a screen as varying brightness dots according to their strength, such that the ultrasound image of the object to be detected is displayed, the basic process of which is illustrated in FIG. 9. The ultrasound image may be obtained by performing a filtering processing, a time gain compensation, an envelope detection, a twice sampling, a logarithmic compression, etc., and a scan conversion on ultrasound RF (radio frequency) signals. It is known to those skilled in the art that the above intermediate processing process does not constitute a limitation to the present disclosure.

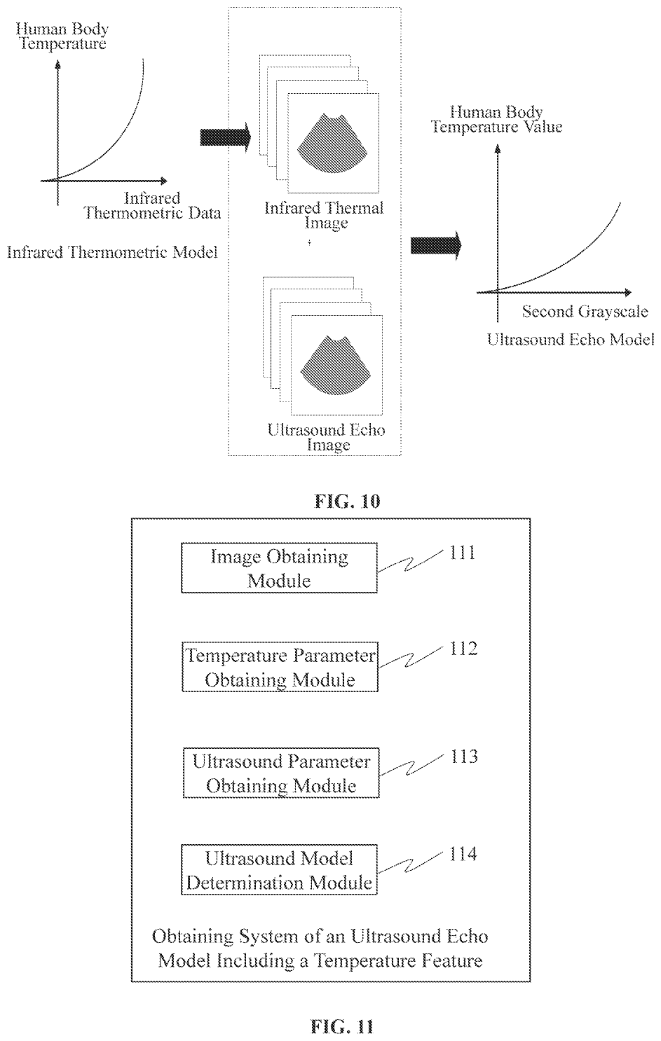

As described in 704, for a second target location of the detected object, the ultrasound echo parameter may be mapped to the temperature parameter by fitting the temperature parameter to the ultrasound echo parameter. In some embodiments, the process of obtaining the ultrasound echo model including the temperature feature may be found in FIG. 10. The ultrasound echo model including the temperature feature may be expressed as a mapping equation, where the independent variable of the mapping equation may be the second gray value of the second pixel point in the ultrasound image, and the dependent variable of the mapping equation may be the first gray value of the first pixel point in the infrared thermal image. The operation 704 may include determining coefficients of the mapping equation by fitting the mapping equation based on the temperature parameters and the ultrasound echo parameters corresponding to a plurality of second target locations of the object to be detected.

In some embodiments, the mapping equation may fit the temperature in the infrared thermometric model and the second gray value of the second pixel point in the ultrasound image, where the horizontal axis expresses the second gray value of the second pixel point and the vertical axis expresses a human body temperature value determined after the fitting. The mapping equation may be y=a₀+a₁x+a₂x²+ . . . +aₖx^{k}, where equation coefficients a₀, a₁, a₂, aₖ are determined by performing a fitting processing on multiple calibrated sets (x, y), and the fitting processing may include, but not limited to, a least squares fitting, an interpolation approximation fitting, etc.

The process for obtaining the ultrasound echo model including the temperature feature of the present embodiment performs a secondary definition of data connotation on the traditional ultrasound detection, that is, the temperature information may be introduced on the basis of the traditional ultrasound detection, and the ultrasound image information including the temperature information of the detected object may be obtained through the organic combination of the infrared thermometric data and the ultrasound echo image, which gives full play to the advantages of the ultrasound echo data detection, while also making full use of the reference role of the infrared thermometry for lesion determination of the detected object, providing the auxiliary determination by combining the relative temperature information on the basis of the traditional lesion or pathological tissue determination, thereby improving the accuracy of the medical diagnosis for the detected object, specifically providing the reliable analysis for the medical diagnosis of the very small or ambiguous lesions and the pathological tissues.

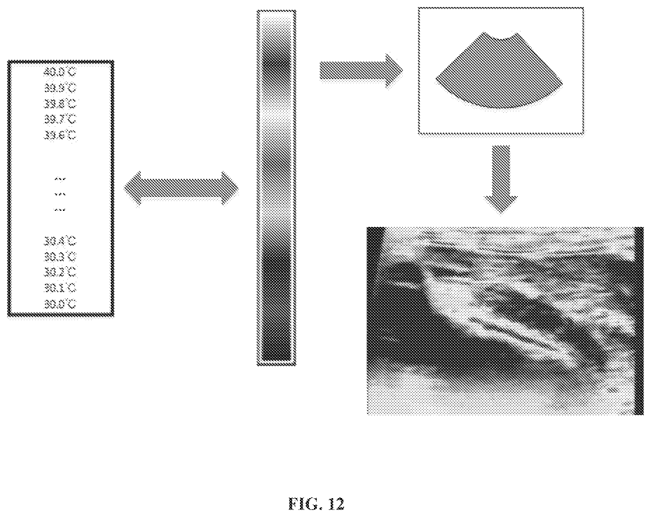

FIG. 11 is a schematic diagram illustrating modules of an exemplary system for obtaining an ultrasound echo model including a temperature feature according to some embodiments of the present disclosure. Referring to FIG. 11, the present disclosure specifically provides the system for obtaining the ultrasound echo model including the temperature feature, including an image obtaining module 111, a temperature parameter obtaining module 112, an ultrasound parameter obtaining module 113, and an ultrasound model determination module 114.

The image obtaining module 111 may be configured to obtain an infrared thermal image and an ultrasound image of an object to be detected.

The temperature parameter obtaining module 112 may be configured to obtain a temperature parameter corresponding to at least one second target location based on a first gray value of a first pixel point corresponding to at least one first target location in the infrared thermal image.

The ultrasound parameter obtaining module 113 may be configured to obtain an ultrasound echo parameter corresponding to the at least one second target location based on the second gray value of the second pixel point corresponding to the at least one second target location in the ultrasound image.

The ultrasound model determination module 114 may be configured to determine an ultrasound echo model based on the temperature parameter and the ultrasound echo parameter corresponding to each second target location. The ultrasound echo model may be configured to map ultrasound echo parameters of different second target locations of the object to be detected to the temperature parameters.

The first target location and the second target location may be determined based on the need for focal analysis of the body tissue, the need for post-fitting processing, or other needs. In some embodiments, the first gray value of the first pixel in the infrared thermal image may be determined according to an infrared temperature model and infrared temperature data corresponding to the object to be detected. The independent variable of the infrared temperature model may be the infrared temperature data and the dependent variable of the infrared temperature model may be the first gray value of the first pixel point. The infrared temperature model may reflect a curve relationship between the original infrared temperature data and the human body temperature, the horizontal axis expressing the infrared signal data and the vertical axis expressing the human body temperature data, and the human body temperature data being the first gray value of the first pixel point of the infrared thermal image. In some embodiments, the mapping equation may be: y=ax²+bx+c, that is, the curve of the infrared thermometric model satisfies part of the needs of the parabolic mathematical definition, where a, b, and c are constants.

In some embodiments, for a second target location of the object to be detected, the ultrasound model determination module 114 may map the ultrasound echo parameters to the temperature parameter by fitting the temperature parameter and the ultrasound echo parameter corresponding to the second target location. In some embodiments, the ultrasound echo model including the temperature feature may be expressed as a mapping equation, where the independent variable of the mapping equation is the second gray value of the second pixel point in the ultrasound image, and the dependent variable of the mapping equation is the first gray value of the first pixel point in the thermal image. The ultrasound model determination module 114 may determine the coefficients of the mapping equation by fitting the mapping equation based on the temperature parameters and the ultrasound echo parameters corresponding to multiple preset target locations of the object to be detected. The temperature parameter may include the first gray value corresponding to the first pixel point in the infrared thermal image. The ultrasound echo parameter may include the second gray value of the second pixel point in the ultrasound image. In some embodiments, the mapping equation may perform a curve-fitting on the temperature in the infrared thermometric model and the second gray value of the second pixel point in the ultrasound image, where the horizontal axis expresses the second gray value of the second pixel point and the vertical axis expresses the human body temperature value determined after the fitting. The mapping equation may be: y=a₀+a₁x+a₂x²+ . . . +aₖx^{k}, where equation coefficients a₀, a₁, a₂, aₖ are determined by performing a fitting processing on multiple calibrated sets (x, y), and the fitting processing includes, but not limited to, a least squares fitting, an interpolation approximation fitting, etc. The human body temperature values may be obtained based on the infrared thermometric model and the infrared thermometric data.

The obtaining system of the ultrasound echo model including the temperature feature of the present disclosure performs a secondary definition of data connotation on traditional ultrasound detection. That is, the temperature information may be introduced on the basis of traditional ultrasound detection, and the ultrasound image information including the temperature information of the object to be detected may be obtained through the organic combination of the infrared thermometric data and the ultrasound echo image, which gives full play to the advantages of ultrasound echo data detection, while also makes full use of the reference role of the infrared thermometry for the lesion determination of the object to be detected, providing the auxiliary determination by combining the relative temperature information on the basis of the traditional lesion or the pathological tissue determination, thereby improving the accuracy of the medical diagnosis, specifically providing the reliable analysis for the medical diagnosis of the very small or ambiguous lesions and the pathological tissues.

FIG. 5 is a flowchart illustrating an exemplary process for ultrasound image processing according to some embodiments of the present disclosure. Referring to FIG. 5, the present disclosure specifically provides the process for ultrasound image processing, including the following operations.

In 501, an ultrasound image corresponding to the object to be detected may be obtained.

In 502, a temperature parameter corresponding to a second pixel point in the ultrasound image may be obtained based on an ultrasound echo model including the temperature feature and a second gray value of the second pixel point.

In 503, the ultrasound image may be updated by assigning a value to the second pixel point based on the temperature parameter.

In 504, the temperature parameter corresponding to the second pixel point is converted into the color level parameter based on the preset pseudo-color level diagram;

In 505, the target image is determined by rendering the updated ultrasound image based on the color level parameter.

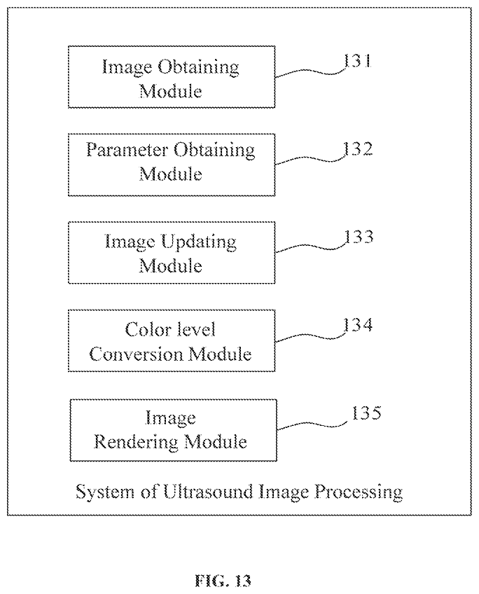

The present disclosure specifically provides a method for ultrasound image processing, which is implemented based on the ultrasound echo model including the temperature feature or the target mapping relationship. The method may include obtaining the temperature parameter by performing a temperature mapping processing on the ultrasound image, assigning the value to the second pixel point in the ultrasound image based on the temperature parameter to overlay and display the temperature parameter on the second pixel point, and finally displaying the ultrasound image with fused, overlaid, and rendered temperature data on the basis of the original ultrasound image. It can be understood that for different objects under inspection, corresponding ultrasound echo models including the temperature feature or the target mapping relationships may be pre-obtained. In the embodiment, the processing may be performed based on the ultrasound echo model including the temperature feature. In 501, the obtained ultrasound image may be of the object to be detected. In 503, the temperature parameter corresponding to each second pixel point obtained in 502 may be assigned to the second pixel point in the form of a grayscale value. In some embodiments, the second pixel points in the ultrasound image and the corresponding temperature parameters may be associated and stored. In 504, the temperature data may be mapped to a pseudo-color level in order to perform a color rendering, and different colors and shades of color may represent different temperatures. FIG. 6 is a schematic diagram illustrating an application of an exemplary process for ultrasound image processing according to some embodiments of the present disclosure. In some embodiments, an ultrasound image obtained from ultrasound-detected data may be combined with an infrared thermal image obtained from an infrared thermography model. The temperature from the infrared thermography model may be fitted with a second grayscale value of the second pixel point in the ultrasound image to obtain the ultrasound echo model including the temperature feature. Subsequently, the obtained ultrasound echo model including the temperature feature may be applied to an ultrasound image to be processed, thereby obtaining the ultrasound image including the temperature data. In some embodiments, the ultrasound echo model including the temperature feature described above may also be obtained by performing a curve-fitting on the first gray value of the first pixel point in the infrared thermal image and the second gray value of the second pixel point in the ultrasound image. In some embodiments, the rendering operation may be performed according to a color level table including correspondences of temperature ranges and colors. The color level table may include a plurality of elements, each of which represents a color. When the rendering operation is performed, a temperature value may be used as an index to look up the color level table to obtain the color corresponding to the temperature value. For example, the color corresponding to a temperature value M may be ColorTable[M]. When the image is finally rendered, the temperature parameter included in each pixel point in the ultrasound image may be converted into the color corresponding to the color level diagram. FIG. 12 illustrates an application effect of the processed ultrasound image. The ultrasound image after rendering was fused with colors including a temperature feature, which can help a user to locate and determine the pathology of the object to be detected in an intuitive, clear, fast, and accurate manner.

The method for ultrasound image processing in the present embodiment is performed based on a redefined ultrasound echo model including the temperature feature for ultrasound image processing. The method may give the play of advantages of ultrasound echo data detection and also make full use of infrared thermometry as the reference role for detecting the lesions of the object to be detected. The method may also combine the relative temperature information with the traditional determination of the lesions or pathological tissues to assist the determination, which improves the accuracy of the medical diagnosis for the object to be detected, specifically providing a reliable analysis basis for the medical diagnosis of the very small or ambiguous lesions and pathological tissues. In addition, a universal temperature thermometric model may be established based on the constancy of the human body temperature, which allows the application in multiple locations, resulting in a relatively higher benefit. The fused relative temperature information may provide an overall display of the temperature distribution of the object to be detected of the patient, achieving a faster initial screening effect relative to the ultrasound image.

FIG. 13 is a schematic diagram illustrating modules of an exemplary system for ultrasound image processing according to some embodiments of the present disclosure. Referring to FIG. 13, the present disclosure specifically provides a system for ultrasound image processing, including an image obtaining module 131, a parameter obtaining module 132, an image update module 133, a color level conversion module 134, and an image rendering module 135.

The image obtaining module 131 may be configured to obtain an ultrasound image corresponding to an object to be detected;

The parameter obtaining module 132 may be configured to obtain a temperature parameter corresponding to a second pixel point in the ultrasound image based on an ultrasound echo model including the temperature feature and a second gray value of the second pixel point;

The image update module 133 may be configured to update the ultrasound image by assigning a value to the second pixel point according to the temperature parameter;

The color level conversion module 134 may be configured to convert the temperature parameter corresponding to the second pixel point into a color level parameter based on a preset pseudo-color level diagram;

The image rendering module 135 may be configured to render the ultrasound image based on the color level parameter.

The present embodiment specifically provides a system for ultrasound image processing, which is implemented based on the ultrasound echo model including the temperature feature, and finally displays the ultrasound image with fused, overlaid, and rendered temperature data on the basis of the ultrasound image. It should be understood that for different objects to be detected, corresponding ultrasound echo models including the temperature feature may be obtained in advance. In the embodiment, the processing may be performed based on the ultrasound echo model including the temperature feature. The ultrasound image obtained by the image obtaining module 131 may also be of the object to be detected. The color mapping module 134 may perform a color mapping of the temperature data based on the temperature ranges and the pseudo-color level diagram for the rendering processing, and different colors and shades of color may represent different temperatures. In some embodiments, the rendering may be performed according to the color level table including correspondences of the temperature ranges and colors. The color level table may include a plurality of elements, each of which represents a color. When the rendering operation is performed, the temperature value may be used as an index to look up the color level table to obtain the color corresponding to the temperature. For example, the color corresponding to a temperature value M may be ColorTable[M]. When the image is finally rendered, the temperature parameter included in each pixel in the ultrasound image may be converted into the color corresponding to the color level diagram.

The system for ultrasound image processing in the present embodiment is performed based on a redefined ultrasound echo model including the temperature feature, which is used to process the ultrasound image. The system may give the play of advantages of ultrasound echo data detection and also make full use of infrared thermometry as the reference role for detecting the lesions of the object to be detected. The system may also combine the relative temperature information with the traditional determination of the lesions or the pathological tissues to assist the determination, which improves the accuracy of the medical diagnosis for the object to be detected, specifically providing a reliable analysis basis for the medical diagnosis of the very small or ambiguous lesions and the pathological tissues. In addition, a universal temperature thermometric model may be established based on the constancy of the human body temperature, which allows the application in multiple locations, resulting in a relatively higher benefit. The fused relative temperature information may provide an overall display of the temperature distribution of the target object to be detected of the patient, achieving a faster initial screening effect relative to the ultrasound image.

The embodiments of the present disclosure also provide an electronic device including a processor, a storage device, and a computer program stored in the memory and operated on the processor. When the processor executes the program, the processor may implement the method for obtaining an ultrasound echo model including the temperature feature as illustrated in FIG. 7 and/or the method for ultrasound image processing as illustrated in FIG. 5.

The electronic device may be represented in the form of a general-purpose computing device. For example, the electronic device may take the form of a server device. Components of the electronic device may include, but not limited to, at least one processor, at least one storage device, and a bus connecting different components (including the storage device and the processor) of the system.

The bus may include a data bus, an address bus, and a control bus.

The storage device may include a volatile memory, such as the random-access memory (RAM) and/or a cache memory. The storage device may further include a read-only memory (ROM).

The storage device may also include a program/utility with a set of (at least one) program modules. The program modules may include, but not limited to, an operating system, one or more applications, other program modules, and program data, each or a combination of which may include an implementation of a network environment.

The processor may perform, by operating the computer program stored in the storage device, various functional applications and data processing, such as the method for obtaining an ultrasound echo model including the temperature feature and/or the method ultrasound images processing as described elsewhere in the present disclosure.

The electronic device may also communicate with one or more external devices (e.g., a keyboard, a pointing device, etc.). The communication may be executed through input/output (I/O) interfaces. A model-generated device may also communicate with one or more networks (e.g., a local region network (LAN), a wide region network (WAN), and/or public networks, such as the Internet) through a network adapter. The network adapter may communicate with other modules of the model-generated device through the bus. The model-generated device may be used in conjunction with other hardware and/or software modules, including, but not limited to, a microcode, a device drive, a redundant processor, an external disk drive array, a RAID (disk array) system, a tape drive, a data backup storage system, etc.

It should be noted that although several units/modules or sub-units/modules of the electronic device are mentioned in the detailed description above, the division is merely exemplary and not mandatory. In fact, according to some embodiments of the present disclosure, the features and functions of two or more units/modules described above may be specified in a single unit/module. Conversely, the feature and function of one unit/module described above may be further divided to be specified by a plurality of units/modules.

The present disclosure provides a non-transitory computer-readable storage medium on which a computer program is stored. The program, when executed by a processor, may implement the method for obtaining an ultrasound echo model including the temperature feature as illustrated in FIG. 7 and/or the method for ultrasound image processing as illustrated in FIG. 5.

The readable storage medium may include, but not limited to, a portable disk, a hard disk, a random access memory, a read-only memory, an erasable programmable read-only memory, an optical storage device, a magnetic memory device, or the like, or any combination thereof.

In possible embodiments, the present disclosure may also be implemented in the form of a program product including program codes. When the program product operates on a terminal device, the program codes may be configured to cause the terminal device to perform a method for obtaining an ultrasound echo model including the temperature feature as illustrated in FIG. 7 and/or a method for ultrasound image processing as illustrated in FIG. 5.

The program codes for executing the present disclosure may be written in any combination of one or more programming languages, and the program codes may be executed entirely on a user device, partially on the user device, as an independent software package, partly on the user device and partly on a remote device, or entirely on the remote device.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by the present disclosure, and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations thereof, are not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the count of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Therefore, by way of example, but not of limitation, alternative configurations of the embodiments of the application may be utilized in accordance with the teachings herein. Accordingly, embodiments of the present application are not limited to that precisely as shown and described.

1. A method for medical image processing, comprising:
obtaining a first image of a target object;
obtaining a target mapping relationship;
obtain a target temperature parameter by performing a first processing on the first image based on the target mapping relationship; and
determining a target image based on the target temperature parameter and the first image.

2. The method for medical image processing of claim 1, wherein the obtaining a target mapping relationship includes:
obtaining thermometric data of a sample object and at least one second image of the sample object, wherein the thermometric data includes a first temperature of at least one first target location;
determining a temperature curve based on the thermometric data; and
determining the target mapping relationship based on the at least one second image and the temperature curve.

3. The method for medical image processing of claim 2, wherein the thermometric data includes an infrared thermal image, and
the determining a temperature curve based on the thermometric data includes:
determining the temperature curve based on a first gray value of a first pixel point corresponding to the at least one first target location in the infrared thermal image, the temperature curve including at least one correspondence between the first temperature and a second temperature corresponding to at least one second target location.

4. The method for medical image processing of claim 2, wherein the first image or the at least one second image includes at least one of an ultrasound image, an MRI image, or a CT image, a type of the first image and a type of the at least one second image being identical.

5. The method for medical image processing of claim 2, wherein the determining the target mapping relationship based on the at least one second image and the temperature curve includes:
determining a correspondence between a second gray value of a second pixel point corresponding to at least one second target location in the at least one second image and a second temperature in the temperature curve; and
determining the target mapping relationship by fitting, based on the correspondence, the second gray value and the second temperature.

6. The method for medical image processing of claim 5, wherein when the at least one second image includes a plurality of second images, the method further includes:
obtaining a plurality of correspondences by determining a correspondence between a second gray value of a second pixel point corresponding to at least one second target location in each of the plurality of second images and a corresponding second temperature in the temperature curve;
determining a plurality of candidate mapping relationships by fitting the second gray value in the each of second image and the corresponding second temperature based on the plurality of correspondences; and
determining the target mapping relationship by performing a second processing on the plurality of candidate mapping relationships.

7. The method for medical image processing of claim 1, wherein the determining a target image based on the target temperature parameter and the first image includes:
determining the target image by performing a third processing on a first pixel point in the first image based on the target temperature parameter.

8. The method for medical image processing of claim 1, wherein the determining a target image based on the target temperature parameter and the first image includes:
obtaining a temperature image based on the target temperature parameter; and
determining the target image based on the temperature image and the first image.

9. The method for medical image processing of claim 1, further comprising:
inputting the target image into a determination model to determine an abnormal region in the target image, wherein the determination model includes a machine learning model.

10. A method for ultrasound image processing, comprising:
obtaining an ultrasound image of an object to be detected;
obtaining a temperature parameter corresponding to a second pixel point corresponding to the ultrasound image based on an ultrasound echo model including a temperature feature and a second gray value of the second pixel point; and
updating the ultrasound image by assigning a value to the second pixel point based on the temperature parameter.

11. The method for ultrasound image processing of claim 10, further comprising:
converting the temperature parameter corresponding to the second pixel point into a color level parameter based on a preset pseudo-color level diagram;
determining a target image by rendering the updated ultrasound image based on the color level parameter.

12. The method for ultrasound image processing of claim 10, wherein the ultrasound echo model including the temperature feature is obtained by:
obtaining an infrared thermal image of a detected object and an ultrasound image of the detected object;
determining a temperature parameter corresponding to at least one second target location based on a first gray value of a first pixel point corresponding to at least one first target location in the infrared thermal image;
determining an ultrasound echo parameter corresponding to each of the at least one second target location based on the second gray value of the second pixel point corresponding to the at least one second target location in the ultrasound image of the detected object; and
determining the ultrasound echo model based on the temperature parameter corresponding to the each of the at least one second target location and the ultrasound echo parameter corresponding to the each of the at least one second target location.

13. The method for ultrasound image processing of claim 12, wherein the ultrasound echo model includes a mapping polynomial; and the determining the ultrasound echo model based on the temperature parameter corresponding to the each of the at least one second target location and the ultrasound echo parameter corresponding to the each of the at least one second target location comprises:
determining a coefficient of the mapping polynomial by fitting the mapping polynomial based on the temperature parameter corresponding to the each of the at least one second target location and the ultrasound echo parameter corresponding to the each of the at least one second target location,
wherein an independent variable of the mapping polynomial is the ultrasound echo parameter and a dependent variable of the mapping polynomial is the temperature parameter.

14. The method for ultrasound image processing of claim 12, wherein
the first gray value of the first pixel point in the infrared thermal image is determined based on an infrared thermometric model corresponding to the detected object and infrared thermometric data corresponding to the detected object; and
the infrared thermometric data is an independent variable of the infrared thermometric model and a dependent variable of the infrared thermometric model is the first gray value of the first pixel point.

15. The method for ultrasound image processing of claim 12, wherein the ultrasound echo parameter is obtained through a beamforming processing based on an ultrasound transmission parameter of the at least one second target location and an ultrasound reception parameter of the at least one second target location.

16. A system for medical image processing, comprising:
at least one storage device including a set of instructions; and
a processing device in communication with the at least one storage device, wherein when executing the set of instructions, the processing device is configured to direct the system to:
obtain a first image of a target object;
obtain a target mapping relationship; and
obtain a target temperature parameter by performing a first processing on the first image based on the target mapping relationship; and
determine a target image based on the target temperature parameter and the first image.

17. The system for medical image processing of claim 16, wherein to obtain the target mapping relationship, the processing device is configured to direct the system to:
obtain thermometric data of a sample object and at least one second image of the sample object;
determine a temperature curve based on the thermometric data;
obtain the target mapping relationship based on the at least one second image and the temperature curve.

18. The system for medical image processing of claim 17, wherein the thermometric data includes an infrared thermal image, and
to determine the temperature curve based on the thermometric data, the processing device is configured to direct the system to:
determine the temperature curve based on a first gray value of a first pixel point corresponding to the at least one first target location in the infrared thermal image, wherein the temperature curve includes a correspondence between a first temperature and a second temperature corresponding to at least one second target location.

19. The system for ultrasound image processing of claim 17, wherein to obtain a target mapping relationship based on the at least one second image and the temperature curve, the processing device is configured to direct the system to:
determine the correspondence between a second gray value of a second pixel point corresponding to the at least one second target location in the at least one second image and the second temperature in the temperature curve; and
determining the target mapping relationship by fitting the second gray value and the second temperature based on the correspondence.

20. The system for ultrasound image processing of claim 16, wherein to determine the target image based on a target temperature parameter and the first image, the processing device is configured to direct the system to:
determine the target image by performing a third processing on a first pixel point in the first image based on the target temperature parameter.
